(19) United States
(12) Patent Application Publication
(10) Pub. No.: US 2026/0193168 A1
(43) Pub. Date: Jul. 9, 2026

(54) METHOD FOR THE PREPARATION OF AN N-ACETYLATED AROMATIC PRIMARY AMINE

(71) Applicant: NOVACYL, ECULLY CEDEX 09 (FR)

(72) Inventors: Joannah N’GOMPAZA-DIARRA, Lyon (FR); Jean-Paul Antoine COMBET, Lyon (FR); Léonard Louis Antoine JACQUEMET, Lyon (FR); Rémy Claude Roger SEIGNEURET, Assieu (FR)

(21) Appl. No.: 19/129,473

(22) PCT Filed: Nov. 16, 2023
(86) PCT No.: PCT/EP2023/082102

§ 371 Date: May 13, 2025

(30) Foreign Application Priority Data
Nov. 17, 2022 (FR) FR2211970

(51) Int. Cl. C07C 231/02; C07C 231/24

(52) U.S. Cl. CPC C07C 231/02; C07C 231/24

(57) ABSTRACT

The present invention relates to a continuous method for the preparation of an N-acetylated aromatic primary amine comprising the steps of:
b) bringing in contact an aromatic primary amine with an acetylating agent selected from acetic anhydride, an acetyl halide and a mixture thereof in an equimolar or sub-molar amount relative to the aromatic primary amine, whereby a reaction stream comprising an N-acetylated aromatic primary amine is obtained, the bringing in contact taking place in the absence of palladium,
c) cooling the reaction stream, whereby the N-acetylated aromatic primary amine crystallizes,
steps b) and c) being carried out continuously. Such method serves to obtain an N-acetylated aromatic primary amine with good purity and good yield.

The present invention relates to a method for the continuous preparation of an N-acetylated aromatic primary amine, in particular an N-acetylaminophenol, preferably paracetamol, also called N-acetyl-p-aminophenol (APAP), N-(4-hydroxyphenyl)acetamide or acetaminophen.

N-acetyl-aminophenol comprises a plurality of regioisomers, including paracetamol. The analgesic and antipyretic properties of paracetamol have made same a preferred active substance for over a century.

The preparation of N-acetyl-aminophenol has been extensively studied. Many methods of synthesis have been described, typically starting from phenol, chlorobenzene or nitrobenzene.

A plurality of methods have proven the effectiveness thereof on an industrial scale. Among such methods, Celanese has developed a path, the last step of which is a Beckmann rearrangement of p-hydroxyacetophenone oxime.

In the other industrial methods, the last step is the N-monoacetylation of p-aminophenol (hereinafter denoted PAP). Monoacetylation of PAP can take place in the presence of acetic anhydride or acetic acid.

Using acetic acid in such reaction involves a high reaction temperature, a high residence time that leads to the production of unwanted impurities in the final product.

Many documents of the prior art also indicate that the use of acetic anhydride would have certain inconveniences, in particular insufficient purity and a coloration of the final product. The subject matter of said documents is thus how to remedy such problem. Mention may be made in particular of U.S. Pat. Nos. 3,042,719; 3,748,358; 3,781,354 or else U.S. Pat. No. 4,264,526.

The presence of impurities, more particularly colored impurities, leads to a long purification and the production of many wastes and effluents.

Moreover, in the prior art, the monoacetylation reaction of PAP takes place in batch mode, which means a limited productivity at the industrial scale.

In the current environmental context, it is necessary to develop a method for the preparation of N-acetylated aromatic primary amine, and preferably N-acetylaminophenol, with a greatly reduced ecological footprint while being industrially viable and having high productivity.

To this end, the subject matter of the invention relates to a method of preparation of an N-acetylated aromatic primary amine, comprising the following steps:
b) bringing in contact an aromatic primary amine with an acetylating agent selected from acetic anhydride, an acetyl halide and a mixture thereof in an equimolar or sub-molar amount relative to the aromatic primary amine, whereby a reaction stream comprising an N-acetylated aromatic primary amine is obtained, the bringing in contact taking place in the absence of palladium,
c) cooling the reaction stream, whereby the N-acetylated aromatic primary amine crystallizes,
steps b) and c) taking place continuously.

The inventors have developed a method of preparation of N-acetyl-aminophenol with a minimized environmental impact.

The continuous implementation of steps b) and c) makes it possible in particular to reduce the quantity of wastes and effluents, while increasing productivity. Not only the acetylation reaction (step b), but also the crystallization (step c), take place continuously.

The aromatic primary amine is preferably an aminophenol, in particular 2-aminophenol, 3-aminophenol, or 4-aminophenol, preferably 2-aminophenol or 4-aminophenol. Particularly preferably, the aromatic primary amine is 4-aminophenol (also called p-aminophenol (PAP)), and the method then serves to prepare paracetamol (APAP).

The acetylating agent is chosen from acetic anhydride, acetyl halide and a mixture thereof. In particular, the acetyl halide is acetyl chloride or acetyl bromide, more particularly acetyl chloride. Typically, the acetylating agent is acetic anhydride.

The method may comprise, before step b), a step a) of supplying the aromatic primary amine.

Preferably, the aromatic primary amine provided in step a) is at a temperature of 0° C. to 120° C., in particular from 0° C. to 110° C., in particular from 5° C. to 90° C. The temperature corresponds to the temperature of the aromatic primary amine just before being brought into contact during step b). Higher temperatures are prevented so as not to degrade the aromatic primary amine.

The degree of purity of the aromatic primary amine is then typically at least 95%, preferably at least 97%, more preferentially at least 98%.

In a first embodiment, the aromatic primary amine supplied during step a) is in essentially in a solvent-free form. Generally, the aromatic primary amine provided is in solid form, typically in powder form.

In such embodiment, the temperature of the aromatic primary amine during step a) is generally from 0° C. to 85° C., preferably from 5° C. to 80° C., more preferentially from 20° C. to 70° C. The temperature corresponds to the temperature of the aromatic primary amine just before the bringing into contact during step b).

In a second embodiment, the aromatic primary amine provided in step a) is in the form of a mixture of the aromatic primary amine and a solvent. The mixture will be used during step b) as a source of aromatic primary amine.

Step a) may comprise a step a1) of mixing an aromatic primary amine and a solvent so as to obtain a mixture of an aromatic primary amine and of a solvent, and then, if appropriate, a step a2) of bringing the mixture to a temperature ranging from 20° C. to 120° C., in particular from 20° C. to 110° C., more particularly from 40° C. to 90° C. The temperature corresponds to the temperature of the mixture just before the bringing into contact during step b). Preferably, the temperature is below the boiling point of the solvent, in particular at least 5° C. below the boiling point of the solvent.

The mixture of an aromatic primary amine and of a solvent may be in the form of a solution or a suspension. The form of the mixture depends in particular on the mass concentration of aromatic primary amine in the solvent and on the temperature of the mixture. In particular, the mixture is a suspension. Alternatively, the mixture is a solution. The solvent may be water, acetic acid or a mixture thereof. The acetic acid/water proportions may vary from 100/0 to 0/100, in particular from 90/10 to 10/90, e.g. from 75/25 to 25/75. More particularly, the solvent is water. Advantageously, the solvent is acetic acid. The concentration by mass of aromatic primary amine in the mixture may be from 10% to 50%, i.e. from 0.10 kg to 0.50 kg of aromatic primary amine in 1 kg of the aromatic primary amine-solvent mixture. Preferably, the concentration is from 15% to 45%, more preferentially from 20% to 45%.

The step a) can take place continuously. More particularly, steps a1) and a2) take place continuously.

The method may comprise, prior to step b), a step a') of providing an acetylating agent selected from acetic anhydride, acetyl halide and a mixture thereof.

Preferably, the acetylating agent supplied in step a') is at a temperature of −10° C. from the boiling point thereof, in particular 0° C. from the boiling point thereof, more particularly 5° C. from the boiling point thereof. When the acetylating agent is a mixture, same is preferably supplied at a temperature less than or equal to the lowest boiling point of the acetylating agents the mixture contains. The temperature corresponds to the temperature of the acetylating agent just before the bringing in contact during step b). Higher temperatures are prevented in order not to degrade the acetylating agent. According to a first alternative, the acetylating agent is at the outside temperature, typically between −10 and 40° C. Indeed, the stock of acetylating agent is generally kept outside. According to a second alternative, the acetylating agent is at the same temperature as the temperature of the aromatic primary amine.

The degree of purity of the acetylating agent is typically at least 95%, preferably at least 97%, more preferentially at least 98%.

In a first embodiment, the acetylating agent provided is in a form which is essentially solvent-free.

In a second embodiment, the acetylating agent provided is in the form of a mixture of acetylating agent and a solvent. The mixture of acetylating agent and solvent will be used during step b) as the source of acetylating agent.

Step a') may comprise a step a1′) of mixing acetylating agent and a solvent to obtain a mixture of acetylating agent and a solvent, and then optionally a step a2′) of bringing the mixture to a temperature ranging from 20° C. to 110° C., more particularly from 40° C. to 90° C. The temperature corresponds to the temperature of the mixture of acetylating agent and a solvent just before the bringing in contact during step b). Preferably, the temperature is below the boiling point of the solvent, in particular at least 5° C. below the boiling point of the solvent.

The mixture of acetylating agent and solvent is in the form of a solution at the temperature at which same is used for carrying out step b).

The solvent is preferably acetic acid.

The mass concentration of acetylating agent in the mixture can be from 20% to 98%, i.e. from 0.20 kg to 1 kg of acetylating agent in 1 kg of the acetylating agent-solvent mixture. Preferably, the mass concentration is from 30% to 95%, more preferentially from 40% to 90%.

The step a′) can take place continuously. More particularly, steps a1′) and a2′) take place continuously.

The method comprises a step b) of bringing in contact the aromatic primary amine with an acetylating agent in an equimolar or sub-molar amount relative to the aromatic primary amine, whereby a reaction stream comprising an N-acetylated aromatic primary amine is obtained, the bringing in contact taking place in the absence of palladium.

The method uses an equimolar or sub-molar amount of acetylating agent relative to the aromatic primary amine. Generally, the number of equivalents of acetylating agent relative to the aromatic primary amine is less than or equal to 1.00, preferably from 0.80 to 1.00, e.g. from 0.90 to 1.00, in particular from 0.95 to 1.00. A continuous control of the amount of acetylating agent serves to obtain a cleaner and less colored product at the end of the method. It is thereby possible to simplify the purification of the N-acetylated aromatic primary N-amine obtained.

In particular during step b), the amount of acetylating agent is equimolar with respect to the aromatic primary amine.

Step b) takes place in the absence of palladium, preferably in the absence of any hydrogenation catalyst, or even in the absence of any metal catalyst in the form of metal or metal oxide. Thereby, there is no hydrogenation reaction at the same time as acetylation, which differentiates the method according to the invention from the methods using a hydrogenation of a nitro-aromatic compound (in particular a nitrophenol, such as para-nitrophenol) to obtain an aromatic primary amine which is acetylated concomitantly. Preferentially, step b) is carried out under pressure.

Preferentially, step b) takes place in a solvent. The solvent can be added during step b). The solvent is typically water, acetic acid or a mixture thereof, more particularly acetic acid. Preferably, when the aromatic primary amine supplied during step a) is in a form which is essentially solvent-free, the method comprises the addition of a solvent during step b). Preferably, when the aromatic primary amine supplied during step a) is in the form of a mixture of the aromatic primary amine and of a solvent, no solvent is added during step b) (the term “no solvent” means no solvent additional to the solvent of the aromatic primary amine/solvent mixture and of the acetylating agent/solvent mixture if the acetylating agent is added in the form of an acetylating agent/solvent mixture).

Bringing in contact the aromatic primary amine with the acetylating agent during step b) leads to the acetylation reaction, and the formation of a reaction stream comprising an N-acetylated aromatic primary amine, which is the acetylation product of the aromatic primary amine. Generally, the acetylation is a monoacetylation and the N-acetylated aromatic primary amine is an N-monoacetylated aromatic primary amine.

Generally, step b) takes place at a temperature from 25° C. to 120° C., in particular 25° C. to 110° C., preferably 30° C. to 100° C., more preferentially from 35° C. to 95° C., e.g. 40° C. to 90° C. Below 25° C., the reaction stream is generally too viscous. Above 110° C., the level of impurities produced is often too high.

Generally, step b) lasts less than 2 hours, in particular less than 1 hour, more particularly from 1 seconds to 45 minutes, more preferentially from 1 minutes to 30 minutes. Typically, the higher the temperature, the shorter the duration of step b).

Step b) is generally carried out in a reactor. The reactor may be a tubular reactor or a continuous stirred-tank reactor. Typically, the reactor is a continuous stirred-tank reactor (commonly referred to as CSTR).

The aromatic primary amine (if appropriate in the form of a mixture with a solvent), the acetylating agent (if appropriate in the form of a mixture with a solvent) and the optional solvent are reactor inlet streams. Within the reactor, the input streams are brought in contact, which leads to the formation of a reaction stream comprising an N-acetylated aromatic primary amine coming from the acetylation reaction. The reaction stream is then removed from the reactor.

The length of time of step b) described hereinabove is typically the residence time in the reactor.

The reaction stream within the reactor or at the reactor outlet may be in the form of a solution or a suspension. When same is in the form of a suspension, the method may comprise, after step b), one or two additional step(s) b1) of heating the reaction stream in order to form a solution, and optionally a step b2) of filtering the reaction stream (with the proviso that it is the filtrate which is used for the rest of the method). When the reaction stream is in the form of a solution, the method may comprise, after step b), an additional step b2) of filtering the reaction stream (with the proviso that it is the filtrate which is used for the rest of the method).

During step b1), the reaction stream can be heated to a temperature ranging from 60° C. to 120° C., preferably from 70° C. to 110° C. More particularly, the reaction stream is heated at least 10° C. more than the temperature of step b), more particularly at least 20° C. more, in particular between 10 and 50° C. more, e.g. between 10 and 30° C. more.

Generally, step b1) lasts less than 2 hours, in particular less than 1 hour, more particularly from 1 seconds to 45 minutes, more preferentially from 1 minutes to 30 minutes. Typically, the higher the temperature, the shorter the length of time of step b1).

Step b1) is generally carried out in a reactor. The reactor may be a tubular reactor or a continuous stirred-tank reactor. Typically, the reactor is a continuous stirred-tank reactor (commonly referred to as CSTR).

The method comprises a step c) of cooling the reaction stream, whereby the N-acetylated aromatic primary amine crystallizes. During the step c), the reaction stream is generally cooled to a temperature from 15° C. to 35° C., in particular from 20° C. to 30° C. Typically, same is at ambient temperature.

Preferably, when, during step b), the acetylating agent is in a sub-molar amount relative to the aromatic primary amine, during step c), acetylating agent is added to the reaction stream. The addition of acetylating agent minimizes the formation of undesirable secondary products and thus improves the purity of the N-acetylated aromatic primary amine obtained using the method. The addition improves the final conversion and limits the coloring.

The amount of acetylating agent added during step c) is preferably such that the total amount of acetylating agent added during steps b) and c) is at least 1.00 molar equivalent, in particular from 1.00 to 1.15 molar equivalents, preferably from 1.00 to 1.08 molar equivalents, particularly preferably of 1.00 molar equivalent, relative to the aromatic primary amine used during step b).

During step c), the acetylating agent is preferably added to the reaction stream when the temperature of said reaction stream is less than 70° C., in particular less than 60° C., preferably less than 55° C., more preferentially less than or equal to 40° C. Typically, the acetylating agent is added to the reaction stream when the temperature of the reaction stream is greater than 15° C. The temperature ranges advantageously make it possible to minimize the formation of undesirable secondary products. Step c) then typically comprises cooling the reaction stream from step b) to a temperature of less than 80° C., in particular less than 60° C., preferably less than 55° C., more preferentially less than or equal to 40° C., and then adding acetylating agent to the reaction stream.

Preferably, the acetylating agent added during step c) is at a temperature from 0 ° C. to 90° C., more particularly from 5 to 50° C. Higher temperatures are prevented in order not to degrade the acetylating agent and/or to not to form undesirable products.

According to a first alternative, the acetylating agent is at the outside temperature, typically between −10 and 40° C. Indeed, the stock of acetylating agent is generally kept outside.

According to a second alternative, the acetylating agent is at the same temperature as the reaction stream at the outlet of step b).

According to a third alternative, the acetylating agent is at the same temperature as the temperature of the reaction stream when the acetylating agent is added. For example, the acetylating agent is at a temperature comprised between 15 and 60° C. when added to the reaction stream, which is as such at a temperature comprised between 15 and 60° C. The degree of purity of the acetylating agent is typically at least 95%, preferably at least 97%, more preferentially at least 98%.

In a first embodiment, the acetylating agent added during step c) is in a form which is essentially solvent-free. Generally, the acetylating agent is in liquid form.

In a second embodiment, the acetylating agent added during step c) is in the form of a mixture of acetylating agent and a solvent. The mixture is then added during step c) as a source of acetylating agent.

Step c) may comprise a step c1) of mixing an acetylating agent and a solvent in order to obtain a mixture of an acetylating agent and of a solvent, then, if appropriate, a step c) of bringing the mixture to the temperature at which same is added during step c). The mixture of acetylating agent and solvent may be in the form of a solution at the temperature at which same is added during step c).

The solvent is preferably acetic acid.

The mass concentration of acetylating agent in the mixture can be from 20% to 98%, i.e. from 0.20 kg to 1 kg of acetylating agent in 1 kg of the acetylating agent-solvent mixture. Preferably, the mass concentration is from 30% to 95%, more preferentially from 40% to 90%.

In a first embodiment, step c) comprises the transfer of the reaction stream into a succession of continuous stirred-tank reactors (CSTRs), more particularly at least two CSTRs, more particularly at least three CSTRs. Preferably, the succession of CSTRs ranges from two CSTRs to ten CSTRs, more preferentially from three CSTRs to eight CSTRs. A succession of CSTRs refers to CSTRs installed in series.

The residence time of the reaction stream in each CSTR is typically from 15 minutes to 1 hour.

In a second embodiment, step c) comprises the transfer of the reaction stream into at least one tubular heat exchanger, preferably from one to thirty tubular heat exchanger(s) in series. The tubular heat exchanger, or each tubular heat exchanger independently of the others where there is a plurality of heat exchangers, may be a tubular heat exchanger with or without a transport system.

The residence time in the tubular heat exchanger or series of tubular heat exchangers is typically 15 minutes to 1 hour.

In a third embodiment, step c) comprises the transfer of the reaction stream into at least one tubular heat exchanger and at least one CSTR, in particular at least one tubular heat exchanger and then at least one CSTR in series. More particularly, step c) comprises the transfer of the reaction stream in a series of one to thirty tubular heat exchangers and then in a series of one to ten CSTRs. The tubular heat exchanger, or each tubular heat exchanger independently of the others where there is a plurality of heat exchangers, may be a tubular heat exchanger with or without a transport system. The residence time in the tubular heat exchanger or in the series of tubular heat exchangers is typically 15 minutes to 1 hour and/or the residence time in each CSTR is typically 15 minutes to 1 hour.

The method generally comprises, after step c), a step of recovery of the N-acetylated aromatic primary amine. The step of recovery of the N-acetylated aromatic primary amine may comprise a step c3) of filtration of the reaction stream and optionally a step c4) of washing the N-acetylated aromatic primary amine with the same solvent as steps b) and c) or with another solvent. Typically, the other solvent is chosen from acetic acid, water or a mixture thereof.

The method may comprise or not comprise an additional purification step of the N-acetylated aromatic primary amine after step c). For example, the method may comprise, after step c), a step d) of recrystallization of the N-acetylated aromatic primary amine, more particularly a recrystallization from water, acetic acid or a mixture thereof, more particularly in water.

Typically, the method comprises only one recrystallization of the N-acetylated aromatic primary amine. Alternatively, recrystallization step d) can be repeated.

Typically, step d) is carried out continuously.

The method generally comprises, after step d), a step of recovery of the recrystallized N-acetylated aromatic primary amine. The step of recovery of the recrystallized N-acetylated aromatic primary amine may comprise a step d1) of filtration and optionally a step d2) of washing the N-acetylated aromatic primary amine with the same solvent as step d) or with another solvent. Typically, the other solvent is chosen from acetic acid, water or a mixture thereof.

The method may comprise, after step c), in particular after step c3) or c4) or after step d), in particular after step d1) or d2) if same are present, a step e) of evaporation of the remaining solvents in the N-acetylated aromatic primary amine.

The N-acetylated aromatic primary amine obtained at the end of the method is in the form of crystals. In the case of APAP, the crystalline structure obtained is the type I polymorph.

Typically, in the case of APAP, at the end of step e), the size of the crystals is such that the D 90 is greater than 200 μm, more particularly greater than 250 μm, more particularly comprised between 250 and 700 μm (determined by laser particle size distribution measurement).

The method may comprise, after step e), a step f1) of grinding the N-acetylated aromatic primary amine and/or a step f2) of screening the N-acetylated aromatic primary amine.

The N-acetylated aromatic primary amine obtained after step c), in particular after step c3) or c4), or after step d), in particular after step d1) or d2), is such that the amount of impurities is less than 0.5% by weight, more particularly less than 0.2% by weight. When the aromatic primary amine is aminophenol, the main impurities are aminophenol (unreacted starting material) and N-acetylated and O-acetylated aminophenol. The amount of aminophenol (starting material) is preferably less than 100 ppm, more preferentially less than 50 ppm. Advantageously, the percentage of N-acetylated and O-acetylated aminophenol is less than 0.1%, e.g. less than 0.05%.

When the N-acetylated aromatic primary amine is APAP, the color thereof is white to off-white.

The invention is illustrated by the following examples.

### EXAMPLE 1

### Invention

4-aminophenol, acetic anhydride and acetic acid are added concomitantly by separate streams in a first CSTR.

4-aminophenol (PAP) is added at ambient temperature at a flow-rate of 3,980 g/h.

Acetic acid is added at ambient temperature (20° C.) at a flow-rate of 7,039 g/h.

0.96 eq. Acetic anhydride (AA) is added at a flow-rate of 3,556 g/h (step b), whereby acetylation occurs to form paracetamol (APAP).

The first CSTR is at a temperature of 80° C. with a residence time of the reaction stream of 1 minute.

The reaction stream is then transferred to a second CSTR which is at a temperature of 100° C. with a residence time of the reaction stream of 10 minutes (step b1).

The reaction stream is filtered (step b2)).

Same is then transferred into a COBR tubular heat exchanger with oscillating counterblade) tubular heat exchanger with a residence time of 20 minutes and an outlet temperature of 25° C. (step c)).

The reaction stream is transferred to a third CSTR.

0.06 eq AA is added to the third CSTR.

The third CSTR is at a temperature of 25° C. with a residence time of the additional reaction stream of 4 hours.

The APAP is then filtered (step c 3)), washed with at least 0.3 vol. of acetic acid and optionally at least 0.5 vol. of water (step c4).

The yield obtained is 70%. The total impurity content is less than 0.2% by weight.

### EXAMPLE 2

### Invention

4-aminophenol, acetic anhydride and acetic acid are added concomitantly by separate streams in a first CSTR.

4-aminophenol (PAP) is added at ambient temperature (20° C.) at a flow-rate of 4,218 g/h.

Acetic acid is added at ambient temperature at a flow-rate of 6,390 g/h.

0.98 eq acetic anhydride (AA) is added at a flow-rate of 3,822 g/h (step b), whereby acetylation occurs to form paracetamol (APAP).

The first CSTR is at a temperature of 80° C. with a residence time of the reaction stream of 18 minutes.

The reaction stream is then transferred to a second CSTR which is at a temperature of 105° C. with a residence time of the reaction stream of 10 minutes (step b 1).

The reaction stream is filtered (step b2).

Same is then transferred into a tubular heat exchanger with a scraped surface with a residence time of 20 minutes and an outlet temperature of 45° C. (step c).

The APAP is then filtered (step c3), washed with at least 0.3 vol. of acetic acid (step c4).

The yield obtained is 65%. The total impurities content is less than 0.2% by weight.

The APAP is then recrystallized in water (step d).

The recrystallization yield is 93%. The total impurities content is less than 0.2% by weight.

### EXAMPLE 3

### Comparison with Acetylating Agent in Excess of Aromatic Primary Amine

4-aminophenol dissolved in acetic acid at 70° C. and acetic anhydride are added concomitantly by separate streams in a CSTR.

1.05 eq acetic anhydride (AA) is added (step b), whereby acetylation occurs to form paracetamol (APAP).

The CSTR is at a temperature of 80° C. with a residence time of the reaction stream of 20 minutes.

The reaction stream is then transferred to another CSTR which is at a temperature of 105° C. with a residence time of the reaction stream of 20 minutes (step b1).

The reaction stream is filtered (step b2).

Same is then transferred into a tubular heat exchanger with a scraped surface with a residence time of 20 minutes and an outlet temperature of 70° C. (step c).

The reaction stream is transferred to a CSTR and cooled to 20° C. The APAP is then filtered (step c 3), washed with at least 0.3 vol. of acetic acid (step c4).

The yield obtained is 72%. The level of total impurities was 0.4% by weight, thus twice as high as the levels in examples 1 and 2 according to the invention.

1-10. (canceled)

11. A method for the preparation of an N-acetylated aromatic primary amine comprising the steps of:
b) bringing in contact an aromatic primary amine with an acetylating agent selected from acetic anhydride, an acetyl halide and a mixture thereof in an equimolar or sub-molar amount relative to the aromatic primary amine, whereby a reaction stream comprising an N-acetylated aromatic primary amine is obtained, the bringing in contact taking place in the absence of palladium,
c) cooling the reaction stream, whereby the N-acetylated aromatic primary amine crystallizes, steps b) and c) being carried out continuously.

12. The method according to claim 11, wherein the aromatic primary amine is an aminophenol.

13. The method according to claim 12, wherein the aromatic primary amine is 2-aminophenol, 3-aminophenol, or 4-aminophenol

14. The method according to claim 13, wherein the aromatic primary amine is 4-aminophenol.

15. The method according to claim 11, wherein in step b) the acetylating agent is in a sub-molar amount relative to the aromatic primary amine, and, during step c), acetylating agent is added to the reaction stream.

16. The method according to claim 15, wherein the amount of acetylating agent added in step c) is such that the total amount of acetylating agent added in steps b) and c) is at least 1.00 molar equivalents, with respect to the aromatic primary amine used in step b).

17. The method according to claim 16, wherein the amount of acetylating agent added in step c) is such that the total amount of acetylating agent added in steps b) and c) is 1.00 to 1.15 molar equivalents with respect to the aromatic primary amine used in step b).

18. The method according to claim 17, wherein the amount of acetylating agent added in step c) is such that the total amount of acetylating agent added in steps b) and c) is 1.00 to 1.08 molar equivalents with respect to the aromatic primary amine used in step b).

19. The method according to claim 18, wherein the amount of acetylating agent added in step c) is such that the total amount of acetylating agent added in steps b) and c) is 1.00 molar equivalents with respect to the aromatic primary amine used in step b).

20. The method according to claim 15, wherein during step c) the acetylating agent is added to the reaction stream when the temperature of said reaction stream is less than 70°C.

21. The method according to claim 11 comprising, after step c), a step d) of recrystallization of a N-acetylated aromatic primary amine in water, in acetic acid or in a mixture thereof.

22. The method according to claim 11, wherein step b):
is carried out at a temperature from 25° C. to 120° C., and/or
lasts less than 2 hours.

23. The method according to claim 11, wherein step b) is carried out in a solvent.

24. The method according to claim 23, wherein the solvent is acetic acid, water or a mixture thereof.

25. The method according to claim 11, wherein step b) takes place in a continuous stirred-tank reactor.
